(19) United States
(12) Patent Application Publication
(10) Pub. No.: US 2007/0238916 A1
(43) Pub. Date: Oct. 11, 2007

(54) Semen catheter improvement for artificial insemination in animals

(76) Inventor: Sheng-Jui Chen, Pyng-Jenn City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/398,618

(22) Filed: Apr. 6, 2006

(51) Int. Cl. A61B 17/43; A61D 7/00

(52) U.S. Cl. 600/33

(57) ABSTRACT

The invention discloses a semen catheter improvement for artificial insemination in animals, comprising a connecting orifice disposed at the head part of flexible catheter, a more rigid cap body fitted over the connecting orifice, a covering member disposed at the inward end of cap body and propping against the head part of catheter and a drain orifice having a plug at the outward end. Using the more rigid cap body that is less prone to deformity and the covering member propping against the head part of catheter for exertion of force when operator pinches it between fingers, operator can insert the drain orifice into the insemination tube in a substantially simple, convenient, fast and accurate manner. More so, such design prevents outflow of semen caused by fingers directly pinching the flexible head part of catheter. In addition, the plug of drain orifice may be snapped off to form an open drain opening and plugged reversely back to close the drain opening. As such, the practice of artificial insemination for animals is made simple and convenient, and free of wasteful semen outflow. The device can also be used for screening of semen.

### BACKGROUND OF THE INVENTION

1) Field of the Invention

The present invention relates to a semen catheter improvement for artificial insemination in animals, more particularly a simple design of semen catheter improvement for artificial insemination in animals that is easy and convenient to use, improves the problem of semen outflow, and allows screening of semen. The present invention is applicable, ideal and inventive.

2) Description of the Prior Art

The general assembly of a conventional semen catheter for artificial insemination in animals is as shown in FIG. 1 and FIG. 2. The catheter comprises a flexible tube body 1 and a drain orifice 2 formed by blow molding. After blow molding, the tail 11 of tube body 1 is open for filling of semen, while the long and thin drain orifice 2 has a closed end 21. After the tube body 1 is filled with semen, its tail 11 is closed by hot melting. As such, animal semen sealed in tube body 1 has stable quality and is protected from contamination. During insemination, the operator first cuts open the closed end 21 of drain orifice 2 to form a drain opening 211 (as shown in FIG. 3). Subsequently, the operator inserts the long, slender artificial insemination tube 3 into the female animal body, and then inserts the drain orifice 2 into the insemination tube 3 via the insertion opening 31 at the back end of insemination tube 3. By squeezing the tube body 1, the semen therein is dispensed into the uterus through the insemination tube 3. As described above, the semen catheter for artificial insemination in animals is formed in one body by blow molding. Thus the entire tube is thin and soft, making it convenient and less strenuous for operator to squeeze it. But after using such catheter design for a long period of time, the operators find the following drawbacks:

1. Because the slender and small-diameter drain orifice 2 is formed by blow molding at the same time as the flexible tube body, it is pliable. When it is inserted into the insertion opening 31 at the back end of insemination tube 3, it is prone to bend, deform, or cause semen blockage due to uneven force exerted.

2. Because the outer diameter of blow-molded drain orifice 2 lacks true roundness (it is often formed in flat shape), there is oftentimes a gap between the drain orifice 2 and the insertion opening 31 at the back end of insemination tube 3 that causes the semen to spill from it, leading to waste.

3. Because the drain orifice 2 and the tube body 1 are formed in one body by blow molding, the head part 12 connecting therebetween is also flexible. When the operator inserts the drain orifice 2 into the insemination tube 3, he often holds and pinches the head part 12 with his thumb and index fingers. Thus in the process of insertion, semen outflow occurs when the head part 12 is squeezed before the drain orifice 2 is completely inserted, resulting in waste of semen.

4. Assuring the quality of animal semen before undertaking artificial insemination can improve the fertilization rate. Most livestock farms would examine the semen for artificial insemination with instrument beforehand. But with a conventional catheter tube just described, the closed end 21 of its drain orifice 2 cannot be reclosed after it is cut open. So to prevent the outflow of semen from the drain opening 211, farms that use this kind of catheter tube usually insert the drain orifice 2 into the insemination tube 3 directly after it is cut open without first examining the viability of semen contained therein. Since screening of semen quality cannot be performed, the success rate of artificial insemination often becomes unpredictable.

Thus the assembly and operation of conventional semen catheter for artificial insemination in animals have some deficiencies that can be improved.

### SUMMARY OF THE INVENTION

The primary object of the present invention is to provide a semen catheter improvement for artificial insemination in animals, characterized in which a connecting orifice is disposed at the head part of blow molded flexible catheter, a more rigid cap body is closely adjoined to the connecting orifice, and disposed with a drain orifice and a covering member propping against the head part of flexible catheter. As such, when operator plans to insert the drain orifice into the insemination tube, the covering member of cap body may be used as the point for exertion of force where the fingers hold and pinch. As the head part of catheter is not squeezed directly, semen outflow is not expected to occur.

Another object of the present invention is to provide a semen catheter improvement for artificial insemination in animals, characterized in which the cap body is made by injection molding, giving the drain orifice more rigidity and better true roundness. As such, when the operator inserts the drain orifice into the insemination tube, the phenomenon of bending, deformation or semen blockage is less likely to occur. In addition, as the drain orifice fits closely to the insemination tube, semen outflow is prevented when it is being dispensed into the insemination tube.

Yet another object of the present invention is to provide a semen catheter improvement for artificial insemination in animals, characterized in which a cap body is configured with a plug at the end of drain orifice. The drain orifice will have a drain opening after the plug is snapped open and become closed again after the plug is placed back reversely. As such, semen in the large number of catheter tubes prepared for artificial insemination can be screened beforehand to ensure its quality so as to boost the success rate and stability of artificial insemination.

### BRIEF DESCRIPTION OF THE DRAWINGS

FIG. 1 is a schematic of a conventional semen catheter with its end opened.

FIG. 2 is a schematic of a conventional semen catheter with its end closed.

FIG. 3 is a schematic of a conventional semen catheter inserted into an insemination tube.

FIG. 4 is a perspective view of the semen catheter according to the invention.

FIG. 5 is an exploded view of the semen catheter according to the invention.

FIG. 6 is a front magnified view of the semen catheter with a cap body according to the invention.

FIG. 7 is a side view of FIG. 6.

FIG. 8 is a schematic showing the plug of cap body as shown in FIG. 6 being snapped off.

FIG. 9 is a schematic showing the plug of cap body as shown in FIG. 8 being removed.

FIG. 10 is a schematic showing the plug of cap body being reinserted according to the invention.

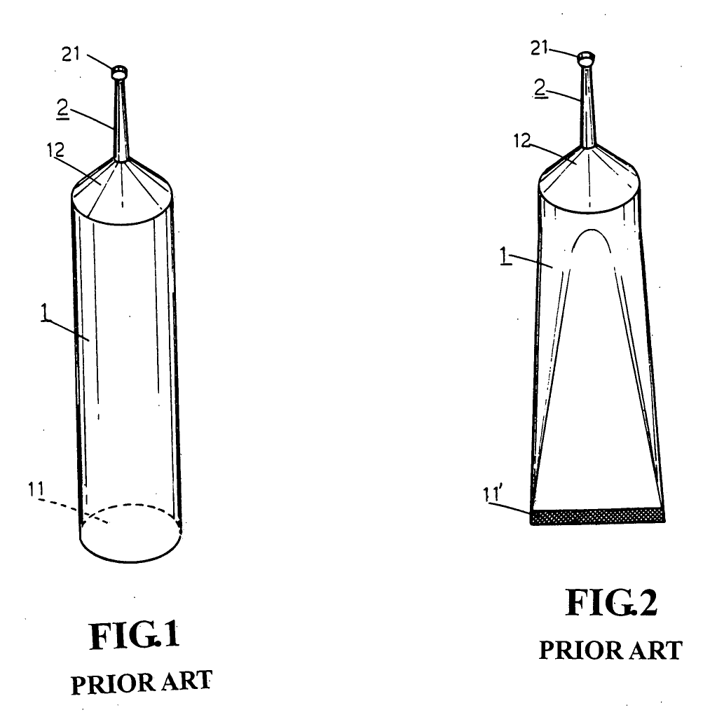

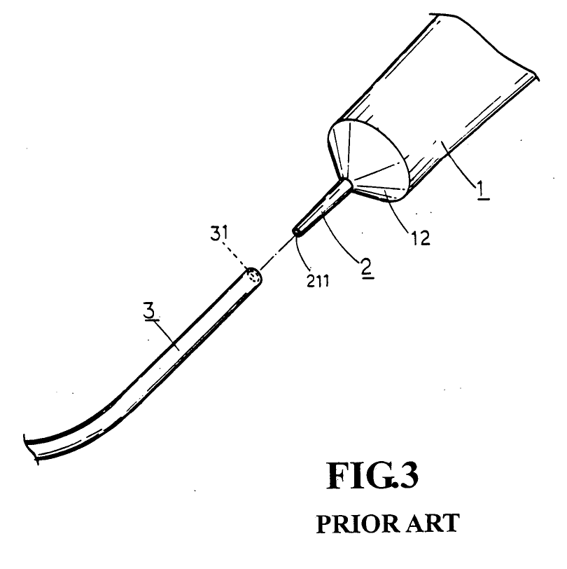

### DETAILED DESCRIPTION OF THE INVENTION

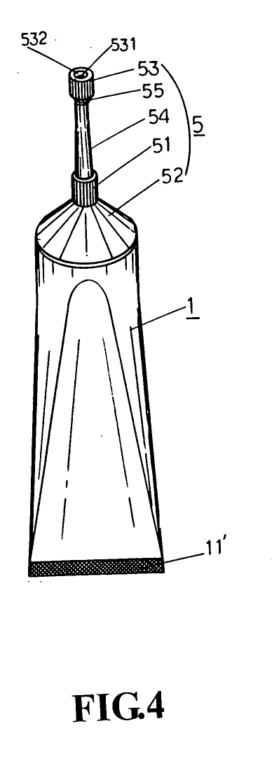

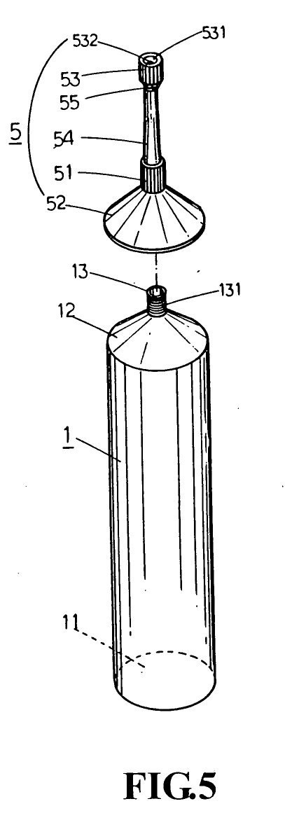

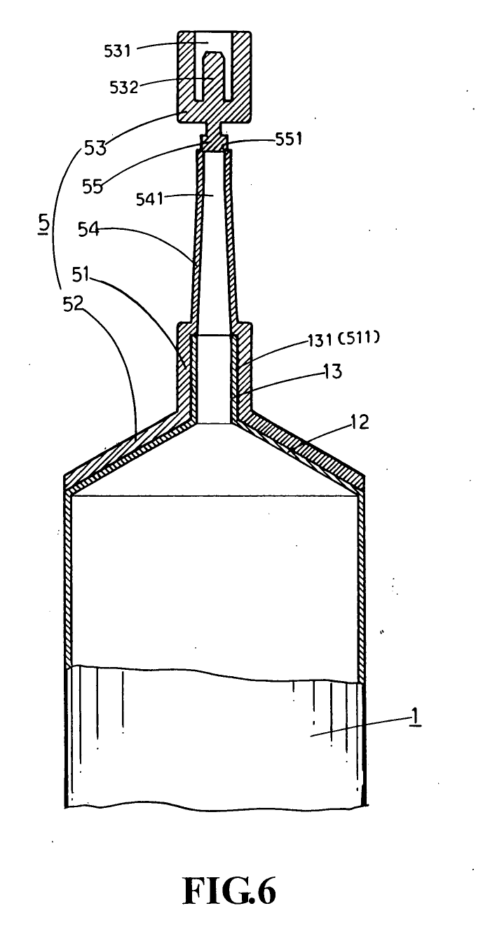

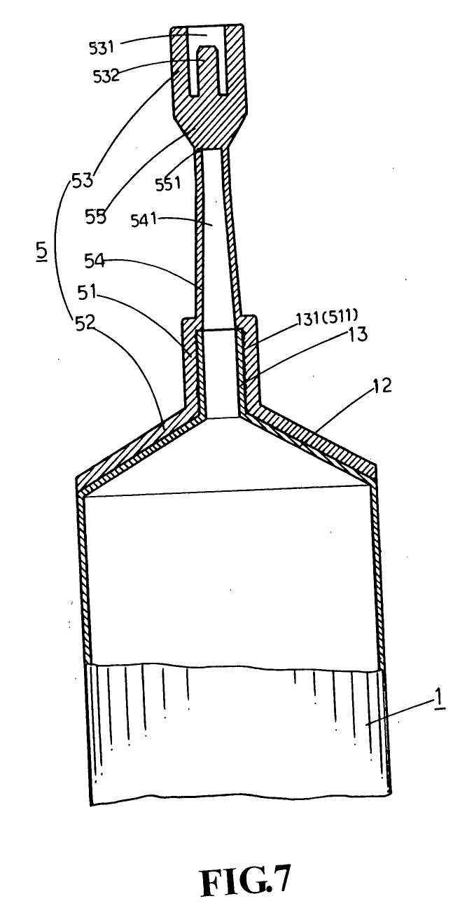

Referring to FIGS. 4, 5, 6 and 7, the semen catheter improvement for artificial insemination in animals according to the present invention comprises a head part 12 on the flexible tube body 1 for accommodating animal semen and a connecting orifice 13 closely adjoined with a more rigid cap body 5. The cap body 5 consists of a sheath member 51 which is closely fitted over the connecting orifice 13 of tube body 1, a covering member 52 disposed at inward end of sheath member 51 which fits exactly over and props against the head part 12 of tube body 1, and a slender drain orifice 54 with a plug 53 at the outward end of sheath member 51.

The drain orifice 54 and plug 53 is connected by a flat and thin extension collar 55. The inward end of extension collar 55 is connected to the inner wall of drain orifice 54 by a little flush member 551 to close the drain track 541 exactly. The outward end of plug 53 is arranged with an accommodation trough 53 of proper depth and having a tenon configured therein. The accommodation trough 531 can be fitted over the outward end of drain orifice 54 with the tenon 532 engaging exactly the tract 541 of drain orifice 54.

The connecting orifice 13 of tube body 1 and the sheath member 51 of cap body 5 can be respectively disposed with spiral threads 131, 511. By coating adhesive over the threads, the engagement between the connecting orifice and sheath member can be made more secure.

The cap body 5 is formed by injection molding and offers more rigidity. Consequently, the slender drain orifice 54 possesses better true roundness.

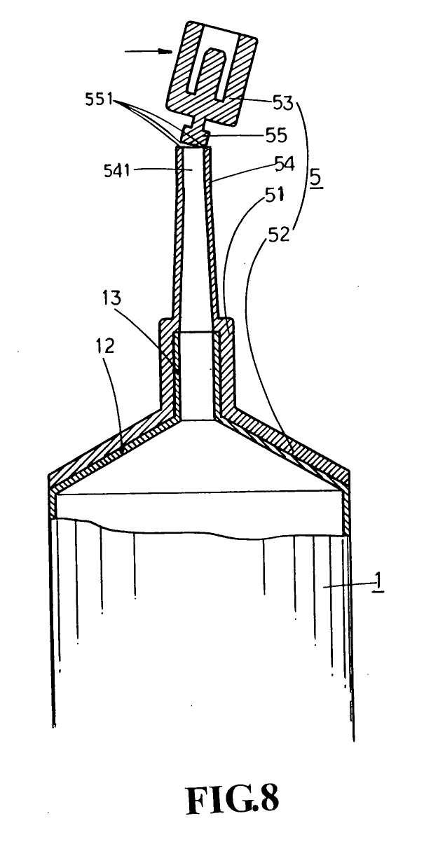

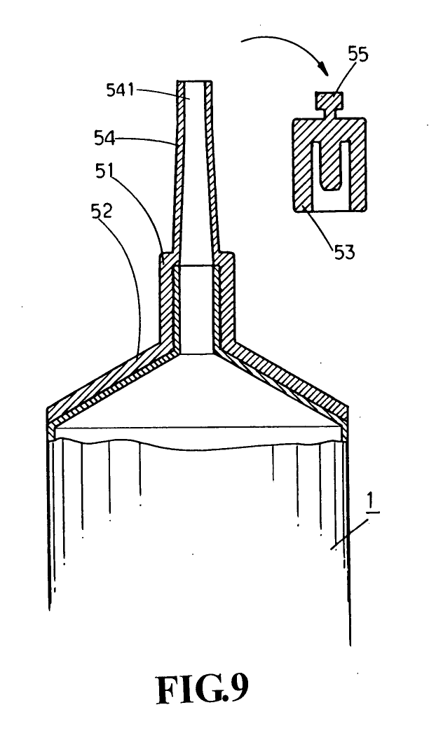

With the aforesaid construction, animal semen can be filled into the tube body 1 from its tail opening 11 and the tail 11′ is then closed by hot melting. Because the connecting orifice 13 at the head part 12 of tube body 1 is closed by cap body 5, the semen inside can retain its quality stability and be protected from contamination. In addition, the extension collar 55 connecting the plug 53 of cap body 5 and the drain orifice 54 is designed with a flat and thin shape that is easy to snap off. Also, the inward end of extension collar 55 is connected to the inner wall of drain orifice 54 by a little flush member 55. Thus when artificial insemination is performed, operator only needs to exert a little force to pull the plug 53 of cap body 5, and the extension collar 55 is snapped off easily at the flush member 551 (FIGS. 8 & 9) to open up the tract 541 of drain orifice 54 for semen to flow out.

Because the cap body 5 connected to flexible tube body 1 is more rigid, and its covering member 52 is fitted over and props against the head part 12 of tube body 1, the slender, yet more rigid drain orifice 54 with plug 53 snapped off is not prone to bend or deform when it is inserted into the insemination tube. Nor will it bend to block the semen flow. More so, the more rigid covering member 52 offers a point of exertion for operator to hold and pinch between fingers. Such design is an effective improvement over conventional flexible catheter head 12 that tends to deform when the fingers exert force and squeeze. Thus this invention allows the practice of artificial insemination in animals to become an easy, convenient, and fast process without wasting any semen.

Again because the cap body 5 is made of more rigid material by injection molding, its drain orifice 54 has better true roundness and fits the insemination tube better. As such, the outflow of semen when it is dispensed into the insemination tube is effectively prevented.

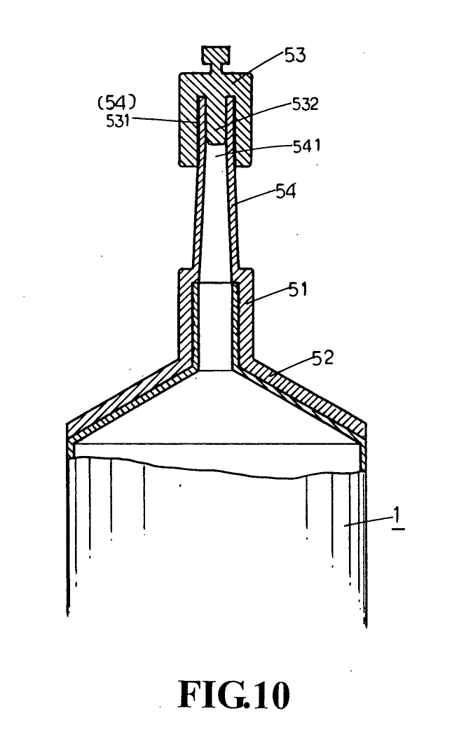

The plug 53 of cap body 5 can be plugged reversely back with accommodation trough 531 fitting over the drain orifice 54 after it is snapped off, and the exact engagement of tenon 532 inside the drain tract 541 (FIG. 10) can prevent the leakage of semen. Before undertaking artificial insemination, animal semen inside the multitude of catheters prepared for the procedure can be sampled by snapping off the plug and inspected for its quality. After sampling, the plug 53 can be plugged reversely back to prevent leakage from the drain tract 541. The screening of animal semen awaiting artificial insemination can assure semen used is of good quality, which can directly increase the success rate of insemination, hence making it a highly efficient and economical procedure.

1. A semen catheter improvement for artificial insemination in animals, characterized by a more rigid cap body closely adjoining the head part of a flexible catheter body, the inward end of cap body being disposed with a covering member covering and propping against the head part of catheter body, and the outward end of cap body being disposed with a drain orifice having a plug.

2. The semen catheter improvement according to claim 1, wherein said flexible head part is disposed with a connecting orifice that communicates with the outside, and a sheath member is disposed between the covering member of the more rigid cap body and the drain orifice that adjoins the connecting orifice closely.

3. The semen catheter improvement according to claim 1, wherein a flat and thin extension collar connects between the drain orifice of cap body and plug, the inward end of said extension collar connects to the inner wall of drain orifice with a little flush member and engages the tract of drain orifice exactly to close it.

4. The semen catheter improvement according to claim 1, wherein the outward end of the plug of cap body is configured with an accommodation trough of proper depth that can fit over the outward end of drain orifice, and a tenon is configured inside said accommodation trough that can engage exactly the tract of drain orifice.

5. The semen catheter improvement according to claim 2, wherein the connecting orifice of catheter body and the sheath member of cap body are respectively disposed spirally fitted threads.

6. The semen catheter improvement according to claim 1, wherein the more rigid cap body is formed by injection molding.

7. The semen catheter improvement according to claim 1, wherein said flexible catheter body is formed by blow molding.
